(19) United States
(12) Patent Application Publication
(10) Pub. No.: US 2025/0311962 A1
(43) Pub. Date: Oct. 9, 2025

(54) METHODS AND DEVICES FOR DETECTING, MEASURING AND GRADING PUPILLARY SHAPES AND IRREGULARITIES

(71) Applicant: Neuroptics, Inc., Irvine, CA (US)

(72) Inventors: Kamran Siminou, Newport Coast, CA (US); Jeffrey W. Oliver, Sugar Land, TX (US); Claudio Privitera, Albany, CA (US)

(21) Appl. No.: 19/071,411

(22) Filed: Mar. 5, 2025

Related U.S. Application Data
(60) Provisional application No. 63/561,840, filed on Mar. 6, 2024.

(51) Int. Cl. A61B 5/00; A61B 3/00; A61B 3/11; A61B 3/12; A61B 5/0205; A61B 5/03

(52) U.S. Cl. CPC A61B 5/4064; A61B 3/0041; A61B 3/112; A61B 3/1241; A61B 5/0205; A61B 5/031; A61B 5/4842

(57) ABSTRACT

A method for assessing pupillary shape using a pupillometer is described. The method includes capturing sequential images of a subject's pupil; analyzing the images using an image processing algorithm associated with the pupilometer to determine pupil shape characteristics, including ellipticity, eccentricity, and cyclotorsion; using an image processing mode associated with the pupilometer selected from speckle imaging and spectral imaging to enhance visualization of vascular structures with an iris of the pupil; comparing detected pupil shape characteristics against predetermined criteria to assess the presence of abnormalities; and outputting a binary status, graphical representation, or quantified measurement of the pupil shape on a display.

This application claims priority from U.S. Provisional Patent Application Ser. No. 63/561,840, filed Mar. 6, 2024, which is incorporated herein by reference.

### BACKGROUND

The present disclosure relates to devices and methods for detecting, measuring and grading pupillary shapes and irregularities in pupillary shapes.

The pupil is the circular aperture located in the center of the eye and which appears as a black circular disk under normal visible ambient light or other indirect illumination. An on-axis illumination source directed through the pupil can be reflected directly off the retina and back through the pupil with reverse illumination that will make it appear brighter than the iris when imaged. The pupil is the component of the eye that allows light to pass through and enter the eye and stimulate the photosensitive cells of the retina. Pupils constrict or enlarge depending on the amount of light present in the environment, and they change in size to allow more or less light to pass through to the retina. In normal and healthy conditions, the pupil is perfectly circular, regardless of its diameter. Irregularly shaped pupils (dyscoria), asymmetrical pupil sizes between left and right eyes (anisocoria) or impairments of the pupil reflex to light stimulation can result from various neurological conditions. Knowing the precise shape and structure can be important for understanding whether the eye is functioning properly, and whether the visual pathway is impaired. It can also help in diagnosing certain medical or neurological conditions such as traumatic brain injury or stroke.

U.S. Pat. No. 9,402,542, which is owned by the current applicants and is incorporated herein by reference in its entirety, taught that a pupillometer can have the ability to detect an irregular shape of a pupil. However, the method of detecting and reporting the shape of a pupil were not considered, and there is therefore a need for an automated pupilometer capable of accurate pupillary shape detection, measurement, and grading. Precise information about pupillary shape and structure delivered in a controlled, reliable manner can provide useful information about the medical condition of a patient.

Therefore, there is a need for a pupillometer that is capable of precise detection, measurement, and grading of pupillary shape and irregularities in pupillary shape.

### SUMMARY

PERRLA (pupils equal round reactive to light and accommodation) has been a popular acronym used in documentation of neurological assessment for many decades [Spector R H. The Pupils. In: Walker H K, Hall W D, Hurst J W, editors. Clinical Methods: The History, Physical, and Laboratory Examinations. 3rd edition. Boston: Butterworths; 1990. Chapter 58].

Cyclotorsion, the rotation of the eye around the visual axis, can occur due to patient posture changes and eye movements. With posture or eye fixation remaining the same between observations, cyclotorsion can indicate changes in tone of the extra-ocular muscles which control pointing direction and the rotation of the eye

The present disclosure provides pupillometers that are able to precisely detect, measure and grade pupillary shapes and irregularities therein to provide accurate assessments of PERRLA, cyclotorsion and other medical conditions relating to the visual pathway or to the neurological condition of patients, relating to various brain disorders, such as brain damage, elevated ICP, brain trauma, traumatic brain injury or disorders, brain tumors, occlusions, strokes, coma, or other neurological disorders as well as monitoring of reactions to drugs and medicine. The pupillometers and methods described herein offer the potential of objective measures of pupillary shape in order to provide information about a patient's medical condition, visual pathway, neurological status, brain disorders or damage, or drug effectiveness or drug reaction.

### BRIEF DESCRIPTION OF THE DRAWINGS

FIG. 1 is an illustration of a method of indicating magnitude and direction of eccentric movement of the pupil within the iris.

FIG. 2 is another illustration of a method of indicating magnitude and direction of eccentric movement of the pupil within the iris.

FIG. 3 is another illustration of a method of indicating magnitude and direction of eccentric movement of the pupil within the iris.

### DETAILED DESCRIPTION

In one embodiment, a pupilometer for taking measurements of a pupil of a subject's eye is described. The pupilometer contains a digital camera for continuously imaging the pupil over a period of time; a microprocessor that includes an algorithm that converts image data representative of the measurements of the pupil into numerical or graphical data representative of the measurements of the pupil taken over time; and a screen that displays an output, wherein the output can include the numerical or graphical data in the form of a display that represents the shape of the pupil of the eye(s) of a subject at a set time or over a period of time. The pupilometer has an infrared light source for illuminating the iris and a stimulating light source to stimulate the pupil of the eye. The algorithm converts image data into a determination of whether the pupil shape is round/circular or not and provides a binary or numeric output of pupil shape status.

The pupilometer can include special imaging modalities of the iris to detect neurological disorders such as traumatic brain injury and stroke. In one example, the image processing mode in the pupilometer is speckle image processing, and blood flow through vasculature in the iris is the primary source of contrast in images. Speckle imaging can leverage a coherent light source (like a laser diode incorporated into the pupilometer) for illumination of a region of interest and can use a sensor array such as a CMOS imager incorporated into the pupilometer to measure local changes of contrast produced by patterns of constructive or destructive interference. These modulation patterns are induced by small changes in the optical pathlength from reflective components of the moving blood, such as circulating white blood cells. A change of optical pathlength resulting from the movement of the blood cell along a vessel equal to one wavelength of the illumination light, will result in a complete cycle of high reflectance (constructive interference) to low reflectance (destructive interference). Using the pupilometer to compare frame by frame reflectance patterns and applying temporal filters allows image contrast enhancement of vascularized areas. In an exemplary pupillometer which utilizes imaging sensors to capture images of the iris, the vascular pattern within the iris is directly spatially correlated to the adjacent pupil edge. Loss of flow to iris vasculature can be a result of microemboli. Microemboli are often associated with embolic or ischemic stroke. Thus, applicants have discovered that the loss of blood flow to iris vasculature can be used as a mircoembolic signal. These signals can indicate embolic strokes which may further be associated with carotid artery stenosis or occlusion of other large artery atherosclerosis. Alternatively, applicants have discovered that a new emergence of an irregular shaped pupil in the absence of loss of regional vascular flow can be an indication of a higher level (i.e. third cranial nerve) neurological disruption. Further, the iris vasculature changes conformation according to the size of the iris. Vessels are straightened and their path becomes less tortuous as the pupil sphincter muscle constricts. Therefore, applicants have discovered that changes in vessel shape, and thus blood flow, can be a more sensitive indicator of neurological disruption to the iris than measuring irregularities in the edge of pupil alone.

In another embodiment of an exemplary pupilometer, the imaging mode of the pupilometer can include spectral imaging of the iris, and the contrast is oxygen saturation of blood contained in iris vasculature. For example, chromophores such as hemoglobin have different optical properties than smooth muscle or melanin. Therefore, illuminating a tissue with wavelengths of light that are preferentially absorbed or reflected in one tissue type over another can result in enhanced contrast of the underlying vasculature or muscular structure. The vasculature and underlying muscle structure are highly correlated with the edge of the pupil. In one embodiment, the source of contrast can be a natural vascular protein biomarker for traumatic brain injury. An example of this type of biomarker is S100B which is a calcium-binding protein produced by astrocytes in the central nervous system that can be released into circulation when the blood-brain barrier is disrupted. S100B has weak intrinsic fluorescence and therefore can be detected circulating in the iris vasculature without the addition of exogenous contrast agents. Thus, in a novel approach of pupillometry, the detection of the S100B biomarker in the iris vasculature using the pupilometer would rely on spectral imaging and fluorescence detection in a method that utilizes the following steps:
1. Illuminating the iris with specific wavelengths—the pupilometer shines light at specific wavelengths that interact differently with hemoglobin, muscle, and melanin. Since S100B has weak intrinsic fluorescence, the system can use fluorescence imaging to detect its presence.
2. Fluorescence Detection of S100B—when excited by the appropriate wavelength of light, S100B fluoresces (emits light at a different wavelength), and the pupilometer captures this emitted light to determine the concentration of S100B in the iris vasculature.
3. Using the Pupil's Edge as a Reference—since the vasculature and muscle structure align with the pupil's edge, this region provides a consistent anatomical reference for imaging. This makes it easier to detect changes in fluorescence due to circulating S100B.
4. Analyzing the Biomarker Signal—the pupilometer can quantify the fluorescence intensity of S100B to determine its concentration. If elevated levels are detected, it could indicate blood-brain barrier disruption, which is a sign of traumatic brain injury (TBI). The iris is used for detection, because the iris is highly vascularized, meaning blood (and biomarkers) pass through it. Unlike blood tests, this method is non-invasive and could provide real-time biomarker monitoring for conditions like TBI and stroke. So, the pupilometer would work by detecting the natural fluorescence of S100B in the blood vessels of the iris, using the pupil's edge as a guide for imaging. This novel method of detecting a neurological disorder has never been used before, and no pupilometers that have this capability currently exist. There may be other vascular proteins which exhibit autofluorescence and which could be indicative of TBI or stroke.

In another embodiment, the source of contrast can be an engineered aptamer which binds to targeted proteins, peptides, carbohydrates, small molecules, or toxins circulating within blood plasma or bound to circulating blood cell membranes to enable or improve contrast of iris vasculature. An example of this would be to use Quantum Dot Labeling where conjugation of, e.g., Intercellular Adhesion Molecule-1 (ICAM-1) antibodies with quantum dots enhances fluorescence-based imaging in circulating blood cells, thus enhancing vascular contrast while simultaneously being a signal of ICAM-1 upregulation due to TBI. In this way, a pupilometer that can detect vascular protein biomarkers of traumatic brain injury or stroke through imaging of an eye can be useful in detecting whether a patient is suffering from traumatic brain injury or stroke. Other vascular protein biomarkers that can be non-invasively detected using this exemplary method and pupilometers are, e.g., VEGF (Vascular Endothelial Growth Factor), VCAM-1 (Vascular Cell Adhesion Molecule-1), MMP-9 (Matrix Metalloproteinase-9), vWF (von Willebrand Factor), TF (Tissue Factor), TM (Thrombomodulin), D-dimer, CRP (C-reactive protein), and HMGB1 (High Mobility Group Box 1), among others.

In another embodiment, a method of pupillary assessment is provided. The method includes using a pupilometer to detect the shape of a pupil, determine whether it's circular/round, and provide a binary or numeric output of pupil shape status. The binary output can be an output indicating whether the shape is round/circular or not.

In another embodiment, a method of using a telecentric lens with a pupilometer is described. The telecentric lens is used for pupil imaging to reduce pupil shape distortion due to angular gaze of the eye with respect to the optical axis of the pupillometer.

In other embodiments, a method of pupillary assessment is provided in which the following pupillary shape characteristics are determined and displayed or reported by a pupillometer:
1. A method of determining the degree of ellipticity, departure from circular shape, of a pupil.
2. A method of scoring the degree of ellipticity in the shape of the pupil.
3. A method of indicating direction (nasal temporal inferior superior) of ellipticity of a pupil.
4. A method to monitor changes in degree or direction of pupil ellipticity
5. A method using image processing in a pupillometer to determine eccentricity, an offset of the center of the pupil with respect to the center of the iris.
6. A method to report the degree (mm or percentage) and direction (nasal temporal inferior superior or combinations and with a measure of angle with respect to fiducial axis).
7. A method using image processing and feature extraction in a pupillometer to measure cyclotorsion of the iris.
8. A method to correct calculation of ellipticity direction resulting from iris rotation of the eye due to cyclotorsion
9. A method to report degree of cyclotorsion in terms of direction or degree.
10. A method to infer changes in extraocular muscle tone [Medial rectus Oculomotor nerve (inferior branch), Lateral rectus Abducens nerve, Superior rectus Oculomotor nerve (superior branch), Inferior rectus Oculomotor nerve (inferior branch), Superior oblique Trochlear nerve (inferior branch), Inferior oblique Oculomotor nerve (inferior branch) from changes in measures of cyclotorsion.
11. A method to infer neurological health from a combination of measures of pupil shape and eccentricity and measure of cyclotorsion.
12. A method to infer changes in neurological health from changes in a combination of measurers of pupil shape and eccentricity and measure of cyclotorsion.

In another embodiment, a method of pupillary analysis of pupillary sector paralysis/dynamic is provided. The method includes image processing and signal analysis to measure dynamic changes in pupil size, shape, eccentricity or cyclotorsion in response to a stimulus, such as a light stimulus, a noxious stimulus (such as an electric shock (e.g., with a peripheral nerve stimulator), or transcranial electromagnetic stimulus of the cortex. Other stimuli that can be used are psychosensory stimulation, such as visual scene changes while maintaining overall scene brightness and field of view.

In another embodiment, a method of pupillary shape analysis is provided. The method includes image processing and signal analysis to measure dynamic changes in pupil size, shape, eccentricity or cyclotorsion in response to a stimulus.

In another embodiment, a method of grading irregularity of shape of a pupil is provided. One embodiment of the method includes providing a pupilometer with a display that displays a graphical overlay indicating areas of the pupil that depart from an expected shape, such as a round/circular shape. The method can include using colors of the overlay to depict the degree of ellipticity. The method displaying on the display the dynamic path tracking of the center of the pupil. The method can also include dynamic cropping of the image of the eye to ensure that the center of the pupil is always displayed at the center of the LCD display, indicating a graphical trace (time course) of the first original frame's center of the pupil compared to the center of the pupil detected in subsequent frames. This method may be further enhanced by identifying, with an overlay, the outline of the outer iris at the sclera boarder. The method can include displaying on the display an arrowhead or other directional indicator indicating primary movement for the direction of eccentricity. The method can include assigning and displaying scalar descriptions of eccentricity. Examples of scalers that can be used:

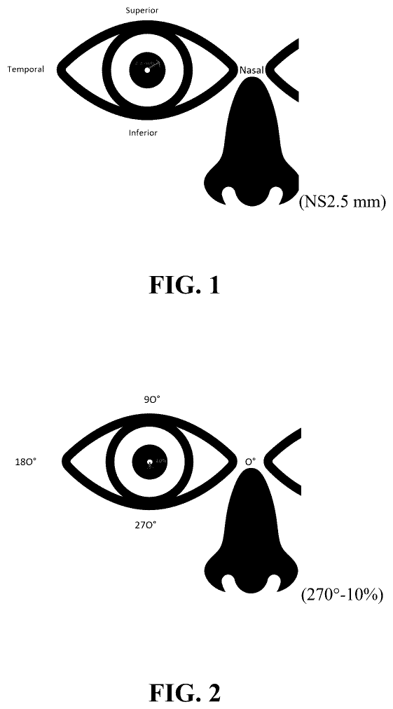

Nasal Superior direction with 2.5 mm eccentric movement as shown in FIG. 1;

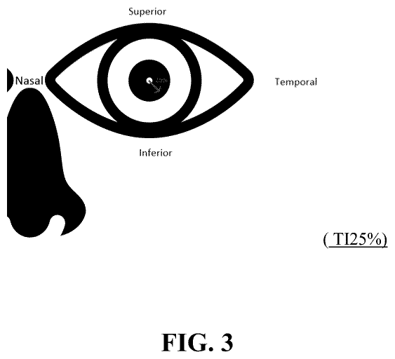

10% of pupil maximum dimension in the 270° direction (i.e. inferior)
as shown in FIG. 2 (10% [symbol] 270°); percent of the Calibrated distance to the iris boarder 25% indicating 2.5 mm eccentric path in the temporal inferior direction with a measured 10 mm diameter measured Iris dimension as shown in FIG. 3
(TI25%) (25% in eye 1.0 mm indicating nasal inferior 1 mm (1 mm [symbol] 270°) for pure nasal 0°. The method can also include grading severity of ellipticity. For example, a grade one deformity can represent 0.1% to 5% deformation, a grade 2, 5.1% to 10% deformity, grade 3, 10.1% to 15% deformity and grade 4 greater than 15% deformity.

In another embodiment, a method using vagal nerve stimulation is provided. The method includes image processing and signal analysis to measure dynamic changes in pupil size, shape, eccentricity or cyclotorsion in response to a stimulus of a vagal nerve. The dynamic response ensures proper treatment placement. The method can also include image processing and signal analysis to measure short-term persistent changes in pupil size and shape in response to a stimulus. This ensures that a proper dose (intensity, frequency and duration) of vagal nerve stimulation has been delivered. The method can also include image processing and signal analysis to measure long-term persistent changes in pupil size, shape in response to a stimulus. This persistent response ensures that a treatment for a disorder has been effective. If not effective, this process can help to determine the need and frequency for further treatments to improve efficacy.

In another embodiment, an integrated pupillometer is described. The pupillometer integrates signals from other biomarkers of medical conditions (e.g., TBI, cardiac arrest, opiate toxicity, substance abuse disorder, Alzheimer's, delirium, diabetes, and etc.). The pupilometer integrates information from, e.g., EEG, EKG, oximeter including NIR spectroscopy, traditional ICP monitor, RF ICP monitor, ultrasound ICP monitor, measures of optic disk/nerve head, blood biomarkers of TBI measured by fluorescent imaging of iris vasculature as described above.

While the invention is susceptible to various modifications and alternative forms, specific examples thereof have been shown by way of example above.

We claim:

1. A pupillometer for assessing pupillary shape and irregularities, comprising:
a digital camera configured to continuously capture images of a subject's pupil over time;
an infrared light source for illuminating an iris and a stimulating light source to elicit pupillary response;
a microprocessor including an algorithm to analyze image data and determine whether the pupil shape is round or irregular, generating a binary output indicative of pupil shape status;
an image processing mode selected from speckle imaging and spectral imaging to enhance visualization of vascular structures within the iris; and
a display screen configured to present an output comprising graphical or numerical representations of the pupil shape and its deviations from an expected form.

2. The pupillometer of claim 1, wherein the algorithm determines and displays on the display the degree of ellipticity of the pupil as a quantified score.

3. The pupillometer of claim 1, wherein the output on the display comprises a graphical overlay indicating areas of the pupil that deviate from an expected shape, with color-coded representation of ellipticity severity.

4. A method for assessing pupillary shape using a pupillometer, comprising:
capturing sequential images of a subject's pupil;
analyzing the images using an image processing algorithm associated with the pupilometer to determine pupil shape characteristics, including ellipticity, eccentricity, and cyclotorsion;
using an image processing mode associated with the pupilometer selected from speckle imaging and spectral imaging to enhance visualization of vascular structures with an iris of the pupil;
comparing detected pupil shape characteristics against predetermined criteria to assess the presence of abnormalities; and
outputting a binary status, graphical representation, or quantified measurement of the pupil shape on a display.

5. The method of claim 4, further comprising tracking changes in pupil shape over time to infer potential neurological conditions.

6. The method of claim 4, wherein said neurological conditions are traumatic brain injury or stroke.

7. The method of claim 4, further comprising using the pupilometer to analyze the relationship between pupil eccentricity and extraocular muscle tone to infer dysfunction in cranial nerves.

8. The method of claim 4, wherein the pupil assessment includes a grading system that categorizes irregularities into severity levels based on measured deviation from a circular shape.

9. The method of claim 4, further comprising the step of using the pupilometer to report the degree of cyclotorsion in terms of direction and angular displacement.

10. A method for correcting pupil shape distortion in a pupillometer, comprising:
employing a telecentric lens to reduce distortions caused by angular gaze of a subject's eye relative to an optical axis of the pupillometer;
processing image data to compensate for rotation of an iris of the subject's eye due to cyclotorsion; and
providing a corrected output displaying true pupil shape characteristics.

11. A method for monitoring pupillary response to vagal nerve stimulation, comprising:
using a pupilometer to capture and analyze pupillary images before, during, and after vagal nerve stimulation;
using the pupilometer to determine changes in pupil size, shape, and eccentricity over time; and
assessing treatment efficacy based on persistent alterations in pupil dynamics.

12. A method for integrating pupillometry with additional biomarkers, comprising:
using a pupilometer to obtain pupillary measurements alongside at least one additional physiological parameter, selected from EEG, EKG, oximetry, intracranial pressure monitoring, or biomarker fluorescence imaging;
correlating pupillary irregularities with external biomarker data to enhance diagnostic accuracy; and
outputting combined diagnostic information to assess neurological or systemic conditions.
